(19) United States
(12) Patent Application Publication
(10) Pub. No.: US 2011/0003310 A1
(43) Pub. Date: Jan. 6, 2011

(54) ORAL FLUID RAPID IMMUNOCHROMATOGRAPHY TEST

(75) Inventors: John Michael Ennis, Vancouver, WA (US); Paul Robert Smith, Washougal, WA (US); Ronald William Mink, Lake, WA (US); James Richard George, Weeki Wachee, FL (US); Toby Devorah Gottfried, Orinda, CA (US); Glen Michael Ford, MOntgomery Village, MD (US)

Correspondence Address: MCDERMOTT WILL & EMERY LLP, 600 13TH STREET, N.W., WASHINGTON, DC, 20005-3096, US

(73) Assignee: BEIJING CALYPTE BIOMEDICAL TECHNOLOGY, Beijing (CN)

(21) Appl. No.: 11/920,747

(22) PCT Filed: May 20, 2005
(86) PCT No.: PCT/CN2005/000701

§ 371 Date: Oct. 28, 2008

(51) Int. Cl. G01N 33/53; B05D 3/10

(52) U.S. Cl. 435/7.1; 427/2.13

(57) ABSTRACT

The present invention relates to an oral fluid rapid immunochromatography test. More particularly, the present invention relates to an oral fluid collection swab separate from a lateral flow immunochromatography strip for detecting an analyte in oral fluid, consisting essentially of a sample pad, a conjugate pad, a test zone and control zone pad made of at least one matrix material, wherein the conjugate pad lies downstream of the sample pad, and is striped with a conjugate; the test and control zone pad lies downstream of the conjugate pad, wherein the test zone is immobilized with an specific binding reagent that specifically binding to the target analyte; and the control zone, downstream of the test zone, is immobilized with a second capture reagent. The invention also relates to a method for manufacturing the strip, a lateral flow immunochromatography method for detecting an analyte in oral fluid by using the strip, and kits containing the strip.

### FIELD OF THE INVENTION

The present invention relates to an oral fluid rapid immunochromatography test. More particularly, the present invention relates to a lateral flow immunochromatography test strip for detecting an analyte in oral fluid, a method for collection of the oral fluid specimen, a method for manufacturing the strip, a lateral flow immunochromatography method for detecting an analyte in oral fluid by using the strip, and kits containing the strip.

### BACKGROUND OF THE INVENTION

Numerous analytical methods have been developed for qualitatively or quantitatively detecting various analytes in tissues and fluids of organisms. Currently most diagnostic testing is done with blood, urine, fecal material, tissue biopsy or oral fluids. Compared with other substances, the collection of oral fluid including saliva and/or oral mucosal transudate for testing entails relatively little invasion of privacy, is relatively safe, and can be accomplished rapidly with relative ease.

To date, many efforts have been done to develop test strips for the collection, transport, and sample handling of oral fluids and to develop oral fluid-based assays, in particular assay for various antibodies and metabolites.

For example, WO 88/07680 disclosed a method for qualitatively or quantitatively detecting the presence or amount of specific IgM, IgG and IgA in the human or animal bodily fluid selected from saliva, tears, semen, urine and cerebro spinal fluid by complicated radioimmunoassay (RIA), enzyme linked immunoassay (ELISA), and the like, which are relatively difficult to perform, time consuming, and costly.

U.S. Pat. No. 5,103,836 disclosed a method of collecting substances from an oral cavity for testing comprising the steps of: inserting an absorbent pad impregnated with salts of a hypertonic solution, wherein the salts of the hypertonic solution are in an effective concentration in the pad to recover a high concentration of said substances, into the oral cavity; removing the pad from the oral cavity; and preserving the pad for subsequent removal of the collected substances from the pad for ELISA. The absorbent pad disclosed in this disclosure need to be pretreated with hypertonic solution to provide high concentration of analytes, and the complicated ELISA is again used to detect the analytes.

U.S. Pat. No. 6,303,081 disclosed a test strip for collecting and transporting aqueous fluid from the oral cavity to a lateral chromatographic strip for testing. The lateral chromatographic strip is placed within and extends along a cavity defined in a housing. At least one inspection site to the lateral chromatographic strip is provided to enable inspection of selected sites on the lateral chromatographic strip for test results. A porous wick material protrudes from the housing to a collection site exterior of the housing at one end and communicates to the lateral chromatographic strip at the other end. The porous wick materials have particulate construction, the particles adsorbing aqueous oral fluid to transport the fluid from the mouth to the lateral chromatographic strip without substantial absorption. The porous wick material readily releases oral fluid to the lateral chromatographic strip. Prevention of reverse flow to the oral cavity from the lateral chromatographic strip naturally occurs due to the circuitous flow path of the porous wick material. A bite plate is coupled to the housing and insertable between the teeth of the patient to position the porous wick in the oral cavity for collecting the oral fluid. The bite plate is typically held in place by the occlusal force of the teeth to position the porous wick in the buccal space. By observing the lateral chromatographic strip while the test strip is in the mouth immediate test result are obtained. US patent application No. 2002/0192839, US patent application No. 2002/0155029 also disclosed improved test strips and methods for one step collection of oral fluid for detection and/or quantification of analytes in the oral fluid, the disclosure of these patent or applications are all incorporated herein by reference in their entirety.

Although the assays by means of above-mentioned test strips have the advantages of direct, rapid, and require no complicated steps, it is inconvenient for the subject to bite the test strip all the time during the course of detection. Furthermore, for assays having “invalid” indicators, where an invalid result requires retesting, or for such diagnostic tests such as HIV that routinely require that a confirmatory test be performed on the specimen collected, the subject will have to bite the test strip for another one or more tests. It should be noted, however, that oral mucosal transudate collection typically reduces the available IgG-rich exudates on the surface of oral tissues for as long as an hour after the initial collection, making followup collections for retesting with “one shop sample collectors/test combinations” inconvenient, since the subject being tested must wait either until the exudates level is higher or risk being tested with a sample that may give reduced sensitivity (R. Mink, unpublished data).

### SUMMARY OF THE INVENTION

The present invention has been made keeping in mind the above disadvantages occurring in the prior arts, and an object of the present invention is to provide a simple, convenient and cost-effective oral fluid collection with a lateral immunochromatography strip suitable for sensitively and rapidly detecting the analytes in the oral fluid.

Another object of the present invention is to provide a method for manufacturing the lateral flow immunochromatography test strip.

Still another object of the present invention is to provide a sensitive method suitable for sensitively and rapidly detecting the analytes in the oral fluid.

Yet another object of the present invention is to provide kits for sensitively and rapidly detecting the analytes in the oral fluid.

Therefore, in the first aspect, the present invention provides a lateral flow immunochromatography test strip for detecting an analyte in oral fluid, consisting essentially of a sample pad, a conjugate pad, a test zone and control zone pad made of at least one matrix material, wherein the conjugate pad lies downstream of the sample pad, and is striped with a conjugate; the test zone and control zone pad lies downstream of the conjugate pad, and contains the test zone and control zone, wherein the test zone is immobilized with an specific binding reagent that specifically binding to the target analyte; and the control zone is immobilized with a second capture reagent.

In an embodiment of this aspect, the analyte to be tested is selected from antibodies against antigens of infectious disease, hormones, growth factors, therapeutic drugs, drugs of abuse and products of the metabolism of drugs of abuse.

In another embodiment, the matrix material is selected from inorganic powders, such as silica and alumina; glass fiber filter paper; natural polymeric material particularly cellulose-based materials, chromatographic paper; synthetic or modified naturally occurring polymers such as nitrocelluse, cellulose acetate, poly(vinyl chloride), polyacrylamide, crosslinked dextran, agarose; and the combination thereof.

In still another embodiment, the matrix material for the sample pad is glass fiber filter paper; the matrix material for the conjugate pad is polyester material; the matrix material for the test pad and control pad is nitrocellulose membrane.

In still another embodiment, the conjugate comprises a label conjugated to a first capture reagent that captures antibodies endogenous to the oral fluid. The label is selected from colloidal gold particles; elemental or metal sol particles including selenium, silver, ferrite or carbon; other bead particles including colored latex, liposomes, and dye particles. Preferably, the label is colloidal gold particles.

In still another embodiment, the first capture reagent is selected from antibodies against IgG, IgM or IgA, protein A, protein G, and concanavalin A. Preferably, the first capture reagent is protein A.

In still another embodiment, the specific binding reagent is selected from antigens of infectious disease, hormones, growth factors, therapeutic drugs, drugs of abuse and products of the metabolism of drugs of abuse.

In still another embodiment, the antigen of infectious disease is recombinant or synthetic peptide representing the immunodominant region of HIV protein, in particular HIV envelope protein selected from gp120 and gp41 of HIV-1 and gp36 of HIV-2.

In yet another embodiment, the second capture reagent is selected from antibodies against IgG, IgM or IgA, protein A, protein G, and concanavalin A, wherein the antibody against IgG is goat anti-human IgG antibody.

In the second aspect, the invention provides a method for manufacturing a lateral flow immunochromatography test strip defined by any one of claims 1-17, comprising: a) striping the conjugate onto the conjugate pad; b) immobilizing the specific binding reagent onto the test zone of the test zone and control zone pad; c) immobilizing the second capture reagent onto the control zone of the test zone and control zone pad; d) blocking each of the pads with blocking agent; and e) aligning the resulting pads in fluid communication relative to each other.

In an embodiment, the conjugate is stabilized in a simple or complex sugar solution before striping, wherein the sugar solution contains sucrose, trehalose, potassium stannate and urea hydrogen peroxide.

In another embodiment, the specific binding reagent is immobilized on the test zone and control zone using a biotin/strepavidin linker.

In still another embodiment, the specific binding reagent is immobilized on the control zone using a biotin/strepavidin linker.

In still another embodiment, the blocking agent contains detergents in nonionic, cationic, anionic and amphoteric forms; sugars including sucrose, fructose; or proteins including bovine serum albumin, whole animal serum, casein and nonfat dry milk.

In yet another embodiment, the whole animal serum is fetal calf serum. In another embodiment, the whole animal serum is avian serum selected from goose serum, turkey serum, and chicken serum.

In the third aspect, the invention provides a lateral flow immunochromatography method for detecting an analyte in oral fluid, comprising: (a) collecting oral fluid with a collector separated from the lateral flow immunochromatography test strip defined by any one of claims 1-17; (b) plunging the collector in a volume of sample buffer to release the oral fluid into the buffer to get the mixture; (c) placing the lateral flow immunochromatography test strip defined by any one of the proceeding claims into the mixture; and (d) determining the validity of the test by observing the presence of the signal in the control zone, and determining the presence of the analyte by observing the presence of the signal in the test zone within 15-60 min from start of the test.

In another embodiment, the collector in step (a) is an untreated polyester swab. such as Texwipe Large Alpha Swab TX714A.

In another embodiment, the sample buffer is potassium phosphate pH 7.2+/−0.2 buffered solution, which further contains 0.15 M sodium chloride, 0.1% Triton X-100, 15% heat inactivated chicken serum, 30 μg/ml Avidin, 0.2% Tween 80, 0.2% Tetronic T-904 and 0.0.285% (active ingredient) ProClin 950.

In another embodiment, the volume of the sample buffer is 1000 μl.

In another embodiment, the method further comprises a step of taking out an aliquot of mixture between step (b) and step (c), wherein the volume of the aliquot is 200 μl.

In another embodiment, the signal is a colored line, such as a reddish line.

In another embodiment, the signal is observed within 20-45 minutes.

In the fourth aspect, the invention provides a kit for detecting an analyte in oral fluid, comprising at least one lateral flow immunochromatography test strip mentioned above. The test strip is closed in a desiccated container. Each kit may optionally include the sample buffer, the oral fluid collector, a package insert providing instruction on the use of the enclosed strips, vials containing a positive and negative control for quality testing the test strip, a timer that may be used to determine when the assay of the invention is complete, and/or a biohazard disposal container.

In a preferred embodiment of this aspect, the sample buffer is potassium phosphate pH 7.2+/−0.2 buffered solution, further containing 0.15 M sodium chloride, 0.1% Triton X-100, 15% heat inactivated chicken serum, 30 μg/ml Avidin, 0.2% Tween 80, 0.2% Tetronic T-904 and 0.0285% (active ingredient) ProClin 950.

In another embodiment, the collector is an untreated polyester swab, such as Texwipe Large Alpha Swab TX714A.

Accordingly, an advantage of the present invention is the design simplicity and low cost of materials by virtue of the absence of a housing such as that in other oral fluid rapid test (see for example U.S. Pat. No. 6,303,081, U.S. Patent Applications 20020155029 and 20020192386) and other rapid lateral flow assays (U.S. Pat. Nos. 6,303,081, 5,935,864, 6,485,982, 6,534,320, 6,352,862, 6,187,598, and 6,027,943). Not only does this enable lower cost of product manufacture, but also it provides for a less complicated manufacturing process, which further reduces costs.

Another advantage of the present invention is that the assay itself is separate from the sample collector (such as in U.S. Pat. No. 6,303,081, U.S. Patent Applications 20020155029 and 20020192839 and U.S. Pat. No. 5,935,864), which enables the user to test the specimen multiple times without an additional collection. This is a particular advantage with such assays as lateral flow immunochromatography assays which have “invalid” indicators, where an invalid result requires retesting, or for such diagnostic tests such as HIV that routinely require that a confirmatory test be performed on the specimen collected, since oral mucosal transudate collection typically reduces the available IgG-rich exudates on the surface of oral tissues for as long as an hour after the initial collection, making followup collection for retesting with “one shop sample collectors/test combinations” inconvenient.

A further advantage of the present invention that the oral fluid rapid immunochromatography strip is extremely compact (e.g. 5 mm×1 mm×75 mm) compared to other oral fluid tests (e.g. U.S. Pat. No. 6,303,081), which provides for reduced shipping and storage cost.

Yet another advantage of the present invention is that the method of oral fluid collection provides sufficient volume for other testing opinions such as confirmatory testing.

A further advantage of the present advantage is that the manufacture is less complicated compared to other oral fluid tests (e.g. U.S. Pat. No. 6,303,081), because no housing must be assembled around the lateral flow chromatography strip, resulting in a reduction in both material costs and process time.

### BRIEF DESCRIPTION OF THE DRAWINGS

FIG. 1 illustrates the procedure of the oral liquid rapid immunochromatography test according to a preferred embodiment of the invention, wherein FIGS. 1-1 to 1-15 illustrate the steps of the test.

FIG. 2 illustrates the three possible results of the oral liquid rapid immunochromatography test according to a preferred embodiment of the invention, wherein FIGS. 2A, 2B and 2C represent negative, positive and invalid result respectively.

### DESCRIPTION OF THE PREFERRED EMBODIMENTS

A better understanding of the present invention may be obtained from the following detailed description of the preferred embodiment described in connection with the accompanying drawings.

In the first aspect, the present invention provides a lateral flow immunochromatography test strip for detecting an analyte in oral fluid, consisting essentially of a sample pad, a conjugate pad, a test zone and control zone pad made of at least one matrix material, wherein the conjugate pad lies downstream of the sample zone pad, and is striped with a conjugate; the test zone and control zone pad lies downstream of the conjugation pad, and is immobilized with a specific binding reagent that specifically binds to the target analyte for the test zone, and is immobilized with a second capture reagent for the control zone.

The sample pad receives the oral fluid sample. The sample pad is typically constructed of a material that exhibits low target antibody retention. In some embodiments the sample pad may also function as a mechanical filter, entrapping any undesirable particulate such as dirt, debris, precipitates, mucus, or blood.

The conjugation pad lies downstream of the sample pad and contains a conjugate comprising a label directly or indirectly coupled to a first capture reagent.

The test zone lies downstream of the conjugate pad, and contains a specific binding reagent that specifically binds the target antibody, whereby the labeled conjugate can be immobilized to the matrix.

The control zone is downstream of the test zone in the flow path. The control zone contains a second capture reagent that capture the antibodies captured by the first capture reagent, and is preferably immobilized within the control zone to form a control line that concentrate any labeled antibody conjugate bound by the second capture reagent.

As used herein, the term “oral fluid” refers to one or more fluids found in the oral cavity individually or in combination. These include, but are not limited to saliva and oral mucosal transudate. It is recognized that oral fluid can comprise a combination of fluids from a number of sources (e.g., parotid, submandibular, sublingual, accessory glands, gingival mucosa and buccal mucosa) and the term oral fluid includes fluids from each of these sources individually, or in combination. The term saliva refers to a combination of oral fluids such as is typically found in the mouth, in particularly after chewing. The term “oral mucosal transudate”, as used herein, refers to fluid produced by the passive diffusion of serum components from oral mucosal interstitial into the oral cavity. Oral mucosal transudate often forms one component of saliva.

As used herein, the term “analyte” is used to refer to a moiety that is to be detected in a particular assay. Analytes commonly detected in the assay of the invention include, but not limited to antibodies against antigens of infectious disease, hormones, growth factors, therapeutic drugs, drugs of abuse and products of the metabolism of drugs of abuse. Particularly preferred analytes include antibodies against antigens of infectious disease, such as HIV, hepatitis, and the like. Antigens can be, but are not limited to antigens such as hepatitis B, and antibodies can be, but are not limited to antibodies to HIV, antibodies to HTLV, antibodies to Helicobacter pylori, antibodies to hepatitis, antibodies to measles, antibodies to mumps, and antibodies to rubella. Therapeutic drugs and drugs of abuse or products of the metabolism of drugs of abuse can be, but not limited to tetrahydrocannabinol, nicotine, ethanol, theophylline, phenytoin, acetaminophen, lithium, diazepam, nortryptyline, secobarbital, phenobarbitol. Hormones can be but not limited to testosterone, estradiol, 17-hydroxyprogesterone, progesterone, thyroxine, thyroid stimulating hormone, follicle stimulating hormone, and luteinizing hormone.

As used herein, a “specific binding reagent” is a reagent that specifically binds to the target analyte, selecting from antigens of infectious disease, hormones, growth factors, therapeutic drugs, drugs of abuse and products of the metabolism of drugs of abuse, as described above.

As used herein, an “antigen” is a substance that, when introduced into a mammal or bird, stimulates the production of an antibody. Preferred antigens of the present invention include human immunodeficiency virus (HIV) proteins, particularly the viral envelope protein gp120 and gp41 of HIV-1, and gp36 of HIV-2.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad immunoglobulin variable region gene. Light chains of antibodies are classified as either kappa or lambda. Heavy chains are classified as gamma, mu, alpha, delta, or epsilon, which in turn define the immunoglobulin classes, IgG, IgM, IgA, IgD and IgE, respectively.

The basic structural unit of the antibody (also known as immunoglobulin) is known to comprise a tetramer. Each tetramer is composed of two identical pairs of polypeptide chains, each pair having one light (about 25 kD) and one heavy chain (about 50-70 kD). The N-terminus of each chain defines a variable region of about 100 to 110 or more amino acids primarily responsible for antigen recognition. The terms variable light, chain (V_{L}) and variable heavy chain (V_{H}) refer to these light and heavy chains respectively.

Antibodies may exist as intact immunoglobulins or as a number of well characterized fragments produced by digestion with various peptidases. Thus, for example, pepsin region to produce F(ab′)₂, a dimer of Fab which itself is a light chain joined to V_{H}-V_{H}1 by a disulfide bond. The F(ab′)₂ may be reduced under mild conditions to break the disulfide linkage in the hinge region thereby converting the F(ab′)₂ dimer into an Fab′ monomer. The Fab′ monomer is essentially a Fab with part of the hinge region (see, Fundamental Immunology, W. E. Paul, Ed., Raven Press, N.Y., 1993 for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies.

As used herein, the term “matrix” refers to an insoluble material capable of supporting fluid flow. Matrix materials may be from natural and/or synthetic sources, bibulous or non-bibulous, fibrous or particulate. Matrices of the invention may be formed as continuous strips of the same material or mixture of different materials that are distributed consistently along a common strip, or inconsistently such as to form zones having different physical or chemical characteristics in different regions of the strip. Alternatively, a series of discrete pads can be formed from the same or different matrix materials, with reagents for the assay being added to each pad. The pad may then be placed in fluid communication with each other to form a continuous flow path. Materials used to construct matrices of the invention may be inert or may react with one or more reagents of the invention, provided that the materials remain insoluble during the practice of the invention as described herein. The matrix materials may be selected for inorganic powders, such as silica and alumina; glass fiber filter paper; natural polymeric material particularly cellulose-based materials such as filter paper, chromatographic paper; synthetic or modified naturally occurring polymers such as nitrocelluse, cellulose acetate, poly(vinyl chloride), polyacrylamide, crosslinked dextran, agarose; and the combination thereof. In a preferred embodiment, the matrix is a nitrocellulose membrane.

In a further preferred embodiment of the invention, the glass fiber filter paper is used as the matrix of a sample pad, polyester material is used as the matrix of a conjugation pad, and nitrocellulose membrane is used as the matrix of a test zone and control zone pad.

“Bibulous” refers the ability of certain absorbent materials to support differential solute migration rates during fluid flow through the absorbent material. Absorbent materials with bibulous properties are therefore capable of chromatographic separation of solutes based on physical and/or chemical properties.

As used herein, the term “label” refers to a detectable atomic or molecular moiety that specifically associates with an analyte either directly or indirectly through an analyte-specific binding partner. Labels of the present invention may be detected physically or chemically. Preferable labels are visible to the naked eye when associated to indicate a positive assay result. Labels of the present invention are selected from colloidal gold particles; elemental or metal sol particles including selenium, silver, ferrite or carbon; other bead particles including colored latex, liposomes, and dye particles. In a preferred embodiment of the invention, the label is colloidal gold particles. These labels are well known in the art, and are described in for example G. Frens, Nature, 241, 20-22 (1973) and U.S. Pat. No. 4,313,734, the disclosures thereof are incorporated herein by reference in their entirety.

“Colloidal gold” used in the present invention refers to a sol of fine gold particles that are capable of remaining in an aqueous suspension indefinitely.

As used herein, a “capture reagent” is any molecule that specifically binds to a target antibody. Capture reagents of present invention are preferably immobilized to the matrix in a defined pattern, typically a line perpendicular to the flow pat. Preferred capture reagents are antibodies against IgG, IgM or IgA; protein A, protein G, or concanavalin A.

As used herein, a “first capture reagent” of the invention is a capture reagent conjugated to a label. Preferably, the first capture reagents are antibodies against IgG, IgM or IgA; protein A, protein or concanavalin A. In a more preferred embodiment, protein A or protein G is conjugated to a label, forming a conjugate.

“Protein A” used in the present invention refers to a highly stable surface receptor produced by Staphylococcus aureus, which is capable of binding the Fc portion of immunoglobulins, especially IgGs, from a large number of species (Boyle, M. D. P. and K. J. Reis. Bacterial Fc Receptors. Biotechnology 5:697-703, 1987). One protein A molecular can bind at least 2 molecules of IgG simultaneously (Sjöquist, J., Meloun, B. and Hjelm, H. Protein A is isolated from Staphylococcus aureus after digestion with lysostaphin. Eur J Biochem 29:572-578, 1972).

“Protein G” used in the present invention refers to a cell surface-associated protein from streptococcus that binds to IgG with high affinity. It has three highly homologous IgG-binding domains (see, Lian, et al., 1992. Journal of Mol. Biol. 228:1219-1234 and Derrick and Wigley. 1994. Journal of Mol. Biol. 243:906-918).

As used herein, a “second capture reagent” is a capture reagent immobilized in the control zone to capture the antibodies captured by the first capture reagent. Suitable second capture reagents of the present invention are antibodies against IgG, IgM or IgA, protein A, protein G, or concanavalin A. In a more preferred embodiment, the second capture reagents include anti-IgG antibodies from species other than the one contributing the oral fluid. Still more preferably, the second capture reagent is anti-human IgG antibody; most preferably, the second capture reagent is goat anti-human IgG antibody.

As used herein, “flow path” refers to the route taken by an oral fluid sample as it passes through a matrix. The flow path is preferably a single route, but may include several routes where each route may support liquid flow simultaneously, sequentially or independently relative to other routes.

“Downstream” refers to the directional flow path of a liquid, through a matrix, away from the point of liquid application.

“Upstream” refers to the directional flow path of a liquid, through a matrix, toward the point of the liquid application.

In the second aspect, the present invention provides a method for manufacturing a lateral flow immunochromatography test strip, comprising: a) striping the conjugate onto the conjugate pad; b) immobilizing the specific binding reagent onto the test zone and immobilizing the second capture reagent onto the control zone of the test zone and control zone pad; c) blocking each of the pads with blocking agent; and e) aligning the resulting pads in fluid communication relative to each other.

Striping the Conjugate onto the Conjugation Pad

The conjugate is deposited in the matrix of the conjugation pad in a manner that allows it to be readily mobilizable in the fluid flow upon contacting with the oral fluid sample. To accomplish this, the matrix of the conjugate pad is made from a spun-bonded polyester. The conjugate is striped with a striping solution onto the pad using, for example, either a contact tip or an aerosol tip. Prior to striping, the conjugate is preferably stabilized. For example, the conjugate may be stabilized in 20% sucrose, 5% trehalose and 0.1% urea hydrogen peroxide.

As the oral fluid sample flows through the conjugation pad, the conjugate is solubilized and joins the fluid flow through the test strip.

Immobilizing the Specific Binding Reagent onto the Test Zone

Specific binding reagents suitable for use in the invention may be obtained from any source including native, chemical synthesis or recombinant production, using methods will known to those of skill in the art. For example, the peptide portion of the preferred SEQ ID No. 1 through 5 may be chemically synthesized using solid-phase peptide synthesis techniques, or recombinantly produced by operably linking a nucleic acid encoding the desired peptide into an expression vector, and expressing the nucleic acid in a suitable host. Once isolated, the peptide may be biotinylated using known techniques.

Suitable antigens may be immobilized to the matrix using any method known to those skilled in the art that does not destroy specific binding of the antigen to the target antibody. Preferably, for recombinant protein antigens, the antigen is immobilized directly onto the matrix without further modification. Preferably, the synthesized peptide antigen is immobilized to the matrix using a biotin/strepavidin linker, most preferably, the antigen is coupled to biotin and complexed with strepavidin prior to coupling strepavidin to the matrix. Coupling strepavidin of the complex to the matrix is typically done prior to blocking, for bibulous matrices, using techniques well known to those of the skill in the art. Preferably coupling is achieved in a solution containing at least a 4:1 ratio of strepavidin binding site equivalents to each biotin moiety, although other ratios such as 0.5:1, 1:1, 2:1, 3:1, and 5:1, among others and all intermediate (fractional) ratios, are also contemplated as being part of the invention. For bibulous matrices, the final complex may simply be applied to the matrix material and dried following by blocking with a suitable blocking agent. Suitable antigens, such as many recombinant proteins, that are of high enough molecular weight to bind to a matrix directly do not have to be attached by way of the biotin/strepavidin linker. The amino acid sequences of exemplary, antigenic peptides suitable for use in the present invention are provided as SEQ ID No:1 through 5.

Immobilization of the antigen to the matrix is preferably performed in a manner that serves to concentrate labeled antibody conjugate that specifically binding to the immobilized antigen. By concentrating labeled antibody conjugate, the signal produced by the label is strengthened, improving sensitivity and minimizing the potential of obtaining an erroneous result.

Immobilizing the Second Capture Reagent onto the Control Zone

The control zone contains a second capture reagent, and is preferably immobilized within the control zone to form a control line that concentrates the label conjugate. Second capture reagents suitable for use in the present invention are immobilized to the matrix using known techniques, including those described above for the immobilized antigen. The second capture reagent is may be immobilized to the matrix using a biotin/strepavidin linker, in which case most preferably, the second capture reagent is coupled to biotin and complexed with strepavidin prior to coupling strepavidin to the matrix, as described above. Preferably, the capture reagents such as goat anti-human IgG F(ab′)₂ can be immobilized on a matrix directly without use of the biotin/strepavidin linker.

Blocking of the Pads with Blocking Agents

Although inherently bibulous matrix materials may be used to make the pads in the present invention, fluid flow through the test strips of the present invention is preferably non-bibulous in nature.

Bibulous materials may be converted to materials which exhibit non-bibulous flow characteristics by the application of blocking agents. These agents may be detergents, sugars, or proteins which can obscure the interactive forces giving rise to the bibulous characteristics. Exemplary protein blocking agents include bovine serum albumin, either per se or in methylated or succinylated form, whole animal sera, such as horse or fetal calf serum, and other blood proteins. A preferred blocking agent is avian serum such as goose or turkey serum, most preferably chicken serum. Other examples of protein blocking agents include casein and nonfat dry milk. Detergent-based blocking agents are selected from nonionic, cationic, anionic and amphoteric forms, with the selection is based on the nature of the matrix that is being blocked. Tween 20 is particularly useful detergent for blocking membranes. Exemplary sugars that may be used as blocking agents include sucrose and fructose.

Application of the blocking reagent may be carried out by treating the pads with a solution of the blocking agent in an effective concentration to dispose of unwanted reactivities at the surface. In general, this treatment is conducted with a blocking solution, such as a protein solution of 1-20 mg/ml protein at approximately room temperature for between several minutes and several hours. The resulting coated material is then permanently adsorbed to the surface by air-drying, lyophilization, or other drying methods.

The use of matrix that is inherently bibulous, but convertible to a non-bibulous flow characteristic, is particularly useful for immobilizing specific binding reagents and second capture reagents. For example, a second capture reagent may be applied to the matrix before the application of blocking agents and can be immobilized in situ. Once the second capture reagent has been immobilized to the matrix, the blocking agent may then be applied.

For example, blocking agents of the invention is applied to the sample pad in amounts sufficient to prevent target antibody interaction with the matrix material during operation of the invention. A particularly advantageous blocking agent for use in the sample pad is avian sera, more preferably chicken sera. In a preferred embodiment, the sample pad impregnated with a solution containing polyvinylpyrollidone, bovine serum albumin, avian sera, borate and/or carbonate buffers (about 0.5 M), and Triton X-100 or Tween-20 detergent. For the sample pad made of glass fiber material, a most preferred sample blocking buffer consists of 40% chicken serum (heat inactivated and sterile filtered), 0.25 M potassium bicarbonate, 0.05 M potassium phosphate dibasic, 0.1% Tween 80, 100 mM potassium stannate and 0.2% urea hydrogen peroxide at pH 8.2 to 8.5. The pad is squeezed to remove excess buffer and the pad is dried overnight at 30° C. An advantage of this approach is increased wettability and wicking action of the sample pad.

The composition and pH of the blocking buffer varies with the type of material of the pad. For example, the conjugate pad made of polyester material is blocked by dipping it in a buffer containing polyvinylpyrollidone, chicken serum, bovine serum albumin, 0.1% urea hydrogen peroxide, 100 mM potassium stannate and carbonate and/or borate buffers. The conjugation pad is then dried at 50° C. and forced air for 120 minutes followed by ambient temperature air drying overnight. For test zone and control zone pad is made of nitrocelluse membrane, a most preferred sample blocking buffer consists of 0.15% bovine serum albumin, 0.075 Tween 20 in potassium buffered at pH 7.8+/−0.1.

In the third aspect, the present invention provides a lateral flow immunochromatography method for detecting an analyte in oral fluid, comprising: (a) collecting oral fluid with a collector separated from the lateral flow immunochromatography test strip; (b) plunging the collector in a volume of sample buffer to release the oral fluid into the buffer to get the mixture; (c) placing the lateral flow immunochromatography test strip as described above into the mixture for about 15-60 min. (d) determining the validity of the test by observing the presence of the signal in the control zone, and determining the presence of the analyte by observing the presence of the signal in the test zone.

The principle of the method mentioned above is provided as follows. The oral fluid is wicked by the matrix and migrated up the assay test strip. It rehydrates a conjugate, for example reddish protein A-colloidal gold reagent on the strip, and IgG in the sample becomes bound to the conjugate to form IgG/conjugate complexes. The IgG/conjugate complexes continue to migrate up the strip, and first encounter the test zone of the test strip containing the specific binding reagent that can be specifically bound by the target analyte, and becomes immobilized in the test zone and a signal, for example a reddish colored line appears. This indicates a reactive or positive result. The absence of a signal in the test zone indicates that the sample does not contain the target analyte and constitutes a non-reactive or negative result. The IgG/conjugate complexes continue to migrate up the assay test strip until it encounters the control zone. The control zone contains a second capture reagent, for example goat anti-human F(ab′)₂ IgG fragments immobilized in a line on the assay test strip. The remaining IgG/conjugate complexes become bound to the immobilized F(ab′)₂ fragments and a signal, for example a reddish colored line appears. The appearance of this signal is evidence that the test functioned properly and contained IgG. A signal will appear in the control zone during the performance of all valid tests, whether or not the sample is reactive or non-reactive for target analyte. The specimen continues to migrate past the control zone into the final absorbent pad, which helps draw the IgG/conjugate complexes through the strip and clear any background color.

In this aspect, the sample collector that can be used for this assay can collect oral mucosal transudate from the mouth, known to have higher diagnostic IgG required by other products (e.g. see U.S. Pat. No. 5,103,836). In fact, in a preferred embodiment, the collector used for this assay is an off the shelf untreated polyester swab, i.e. Texwipe Large Alpha Swab, TX714A (Texwipe Inc., Upper Saddle River N.J.).

In an embodiment of this aspect, the sample buffer used to dilute the oral fluid is potassium phosphate pH 7.2+/−0.2 buffered 0.15 M sodium chloride, 0.1% Triton X-100, 15% heat inactivated chicken serum, 30 μg/ml Avidin, 0.2% Tween 80, 0.2% Tetronic T-904 and 0.0285% (active ingredient) ProClin 950. The volume of the sample buffer use to dilute the oral fluid is 1000 μl.

In another preferred embodiment, the method further comprises a step of taking out an aliquot of the mixture between step (b) and step (c). In a particular embodiment, the volume of the aliquot is 200 μl.

When operating correctly, second capture reagent will continue to bind all labeled antibody conjugate until the unbound labeled antibody conjugate is depleted, or the second capture reagent is saturated. As even oral fluid sample from healthy mammals contain endogenous IgG, and the molar amount of labeling agent coupled to label preferably exceeds the molar amount of immobilized antigen, labeled antibody conjugate should always be available to bind to second capture reagent, producing a signal at the control line. Therefore, failure to detect a signal at the control line is indicative of either the absence of sufficient human IgG to produce a visible line, a faulty test strip or poor operation of the strip.

Typically label signals may be observed between 15 and 60 minutes, more preferably between 15 and 45 minutes, most preferably between 15 and 30 minutes after the test strip in inserted into the oral fluid. Reading test results sooner than 15 minutes or later than 60 minutes after the start of the test may give erroneous results. Signals produced by colored labels, as described above, can generally be detected directly from the test strip without further processing. Fluorescent label may require a fluorimeter to detect. Signals produced by metal sol labels may be enhanced using silver salt solution in methods well known to those skilled in the art. Similarly, when enzymes are used, the labels must be contacted with a substrate of the enzyme label that produces a detectable product. Thus these enhanced methods deviate from the routine, single-step assay performed with the colored particulate labels and sols, as the matrix must be contacted with a developing solution (a silver salt or substrate solution) before the label is detected.

In a most preferred embodiment, the present invention provides a test strip containing either recombinant proteins or synthetic peptides representing the immunodominant regions of the HIV-1 gp41 and HIV-2 gp36 envelope proteins and a goat anti-human IgG F(ab′)₂ fragment antibody capture procedural control immobilized onto the nitrocellulose membrane in the test zone and the control zone, respectively.

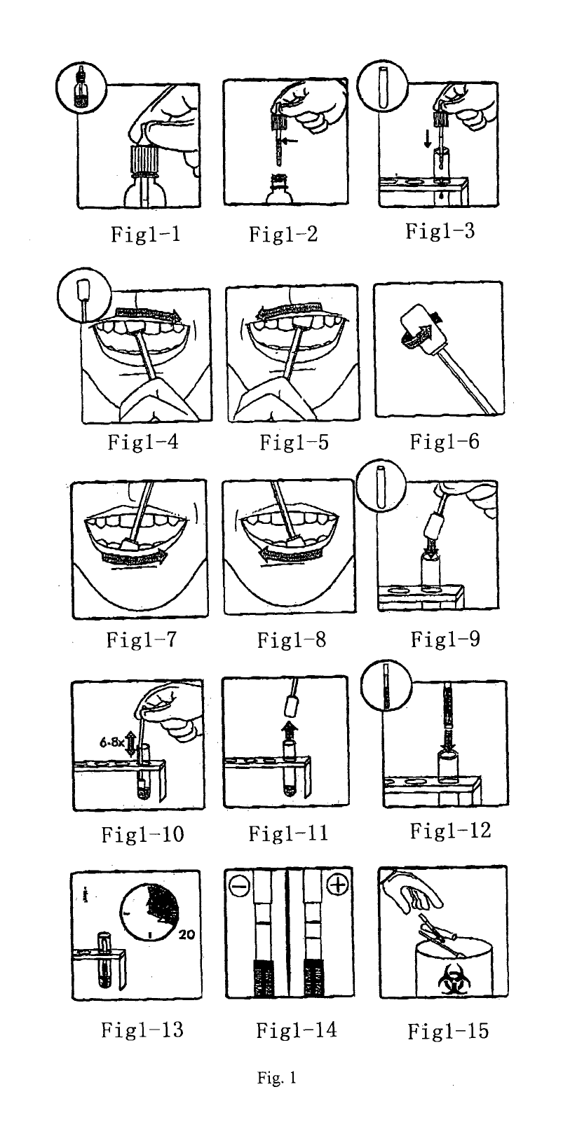

FIG. 1 illustrates the procedure of the oral liquid rapid immunochromatography test according to a preferred embodiment of the invention, wherein FIGS. 1-1 to 1-15 illustrate the steps of the test. To perform the assay, the upper and lower gums of the subjects are swabbed with a polyester swab which is then placed in approximately 1000 μl of oral fluid sample buffer (potassium phosphate pH 7.2+/−0.2 buffered 0.15 M sodium chloride, 0.1% Triton X-100, 15% heat inactivated chicken serum, 30 μg/ml Avidin, 0.2% Tween 80, 0.2% Tetronic T-904 and 0.0285% (active ingredient) ProClin 950) in a test tube and mixed in the test tube. The liquid in the swab is expressed out and discarded, and 200 μl of this specimen mixture is transferred to a clean test tube to perform the assay. The assay test strip is placed vertically into the test tube containing the 200 μl specimen mixture. As the diluted specimen migrates up the assay test strip, it rehydrates a reddish protein A-colloidal gold reagent on the strip, and IgG in the specimen becomes bound to the Protein A/colloidal gold particles to form IgG/conjugate complexes. The IgG/conjugate complexes continue to migrate up the strip, and first encounters the test zone of the assay test strip containing the HIV antigen binding to the anti-HIV antibodies and become immobilized at the antigen line in the test zone and a reddish colored line appears. This indicates a reactive or positive result. The intensity of the line is not proportional to the amount of antibody present in the specimen. The absence of a colored line in the test zone indicates that the specimen does not contain anti-HIV antibodies. The IgG/conjugate complexes continue to migrate up the assay test strip until it encounters the control zone. The control zone contains goat anti-human F(ab′)₂ IgG fragments immobilized in a line on the assay test strip. The remaining IgG/conjugate complexes become bound to the immobilized F(ab′)₂ fragments and a reddish colored line appears. The appearance of the control line is evidence that the test functioned properly and contained IgG. A reddish control line will appear in the control zone during the performance of all valid tests, whether or not the sample is reactive or non-reactive for antibodies to HIV-1 or 2. The specimen continues to migrate past the control zone into the final absorbent pad, which helps draw the IgG/conjugate complexes through the strip and clear any background color. The test results are interpreted after 20 minutes but not more than 45 minutes after the introduction of the assay test strip to the diluted specimen. Reading test results sooner than 20 minutes or later than 45 minutes after the start of the test may give erroneous results. The remaining diluted sample can be used for other test, such as a confirmatory test.

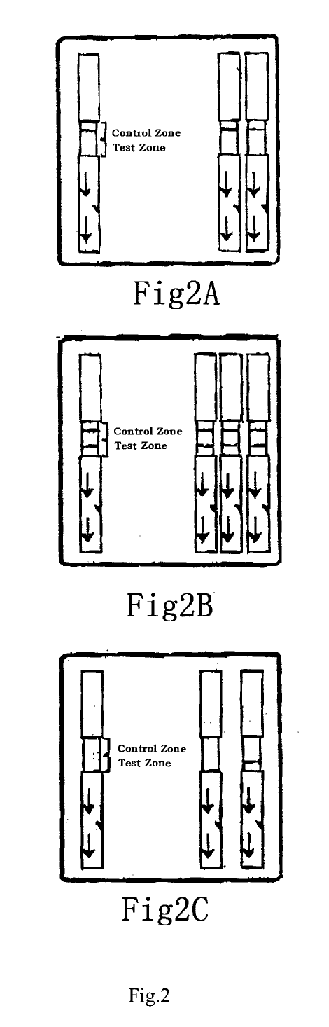

FIG. 2 illustrates the three possible results of the oral liquid rapid immunochromatography test according to a preferred embodiment of the invention, wherein FIGS. 2A, 2B and 2C represent negative, positive and invalid result respectively.

FIG. 2A shows non-reactive results, in which only a single line appears in the control zone, suggesting the absence of reactive anti-HIV-1 or anti-HIV-2 antibodies in the oral fluid sample. The test result is interpreted as a negative for HIV antibodies.

FIG. 2B shows the reactive result, in which both a test and control line appear, i.e. two lines appear on the test strip, in the test zone and control zone, respectively. One of these lines may be darker than the other. A reactive result means that anti-HIV-1/2 antibodies have been detected in the oral fluid sample. This test result is interpreted as a preliminary positive for HIV antibodies.

FIG. 2C shows the invalid results, in which there is no control line in the control zone. The result is invalid even if a test line appears in the test zone. An invalid test should be repeated with a new test strip.

The embodiment described above can be designed in alternative ways that include alternate labels other than gold sol. For example, other labels include but not limited to elemental or metal sols such as selenium, silver, ferrite or carbon, other bead particles such as colored latex, liposomes, and dye particles. Other first capture reagent capable of specifically capturing antibodies in the oral fluid samples include but not limited to antibodies against IgM or IgA; protein G, or concanavalin A. The second capture reagent can also be formulated to include, but not limited to alternate ligands such as protein A or protein G.

In the fourth aspect, the present invention provides kits for detecting an analyte in oral fluid, comprising single used lateral flow immunochromatography test strip as described above. The test strip is closed in a desiccated container. Each kit may optionally include the sample buffer, the oral fluid collector, a package insert providing instruction on the use of the enclosed strips, vials containing a positive and negative control for quality testing the test strip, a timer that may be used to determine when the assay of the invention is complete, and/or a biohazard disposal container.

In a preferred embodiment of this aspect, the sample buffer is potassium phosphate pH 7.2+/−0.2 buffered solution, further containing 0.15 M sodium chloride, 0.1% Triton X-100, 15% heat inactivated chicken serum, 30 μg/ml Avidin, 0.2% Tween 80, 0.2% Tetronic T-904 and 0.0285% (active ingredient) ProClin 950.

Although the foregoing invention has been described in some detail by way of illustration and example for clarity and understanding, it will be readily apparent to one of ordinary skill in the art in light of the teaching of this invention that various changes and modifications may be made thereto without departing from the spirit and scope of the appended claims.

In addition to the diagnosis of HIV by the detection of HIV antibodies in oral fluid, the present invention can be readily configured for the diagnosis of a number of conditions requiring the immunological detection of analytes in oral fluid. It is particularly easy to use the concept of the invention for sexually transmitted disease detection such as syphilis antibody and hepatitis virus antibody.

### EXAMPLES

The following examples will illustrate the invention without limiting it thereto.

### Example 1

### Manufacturing of the Immunochromatography Test Strips

Test strips for rapid HIV-1/2 oral fluid antibody test are provided in this example, wherein glass fiber material is used as the matrix of the sample pad, polyester material is used as the matrix of the conjugation pad, and nitrocellulose membrane is used as the matrix of the test and control pad.

1 inch S & S S-33 glass fiber material is soaked with the blocking buffer consisting of 40% normal chicken serum (heat inactivated), 0.25 M potassium bicarbonate, 0.05 M potassium phosphate dibasic, 0.1% Tween 80, 100 mM potassium stannate and 0.2% urea hydrogen peroxide at pH 8.2 to 8.5, dried at room temperature (15-30° C.) in a low humidity room for 8 hours, then overnight in a desiccated container at 50° C., and kept desiccated.

The conjugate pads was prepared from polyester membrane by striping protein A gold conjugate onto the pad using an aerosol tip. Prior to striping, the conjugate was stabilized in 20% Sucrose, 5% Trehalose, 100 mM potassium stannate and 0.1% urea peroxide. The pad was then dipped in a buffer containing polyvinylpyrollidone, chicken serum, bovine serum albumin, and carbonate buffer and dried at 50° C. using forced air for 50 minutes.

HIV antigens can be coupled to the test zone pads include using a strepavidin/biotin linkage. For the striping, synthetic HIV-1 peptide and synthetic HIV-2 peptide, (e.g. SEQ ID No: 1 to 5) are used at 300 ng/test strip and 0.15 ng/test strip applied to the test pad, respectively. The solution consisting of 1.2 mg/ml HIV-1, 0.06 mg/ml HIV-2, 4.36 mg/ml Avidin and 0.05% isopropyl alcohol is used in the stripping of the test pad. 1 mg/ml goat anti-human IgG Fc F(ab)2 is applied to the control pad.

For the test strip using recombinant HIV-1/recombinant HIV-2, gp41 protein at 0.4-0.7 μg/strip and gp36 protein at 0.04-0.08 μg/strip in 0.001% Tween 80, 5% sucrose and 2% methanol are applied to the test zone, and goat anti-human IgG F(ab′)₂ at 0.175 μg/strip in 5% sucrose, 2% methanol and 0.01 M potassium carbonate at pH 8.4 is applied to the control zone.

The test zone and control zone pad treated as above are blocked by the blocking agent consists of 0.15% bovine serum albumin, 0.075% Tween 20 in potassium phosphate buffer at pH 7.8+/−0.1. Then the resulting pads were aligned in fluid communication relative to each other, with the conjugate pad being downstream of the sample pad; the test and control zone pad being downstream of the conjugate pad such that the control zone is downstream of the test zone.

### Example 2

### Oral Fluid Rapid Immunochromatography Test

Oral liquid rapid immunochromatography test using the strip of the invention is provided, as shown in FIG. 1-1 to FIG. 1-15. Firstly, Mix the sample buffer (potassium phosphate pH 7.2+/−0.2 buffered 0.15 M sodium chloride, 0.1% Triton X-100, 15% heat inactivated chicken serum, 30 μg/ml Avidin, 0.2% Tween 80, 0.2% Tetronic T-904 and 0.0285% (active ingredient) ProClin 950) by gently inverting the bottle about 3 times. Remove the cap from the bottle (FIG. 1-1) and fill buffer to line in dropper (FIG. 1-2). Dispense all of the contents of the dropper into the test tube (FIG. 1-3). Then remove one of the clean swabs provide from the bag. Grasp the swab by the handle. Avoid touching the cloth end of the swab. Subsequently, apply moderate pressure while gently swabbing the upper gum line back and forth with the cloth end of the swab. Begin at one corner of the mouth, swabbing gently and slowly until reaching the other corner of the mouth (FIG. 1-4) and then swab back across the upper gum line to where the start point (about 5-6 second) (FIG. 1-5). Then turn the swab to use the other side of the swab for the lower gums (FIG. 1-6). Using the other side of the swab, gently and slowly swab the lower gum line back and forth. Begin at one corner of the mouth (FIG. 1-7), ending at the other corner of the mouth and then swab back across the lower gum line to where you started (about 5-6 seconds) (FIG. 8). Immediately place the swab in the tube containing the sample buffer (FIG. 1-9).

Grasp the swab handle firmly. Plunge the swab in the sample buffer tube up and down 6-8 times, rubbing both side of the swab against the sides of the tube (FIG. 1-10). Remove the swab from the tube (FIG. 1-11). The sample is now ready for testing. Transfer 200 μl of this sample to an empty test tube. This aliquot will be tested with the assay test strip below. If more than one test strip is to be run on the sample, multiple 200 μl aliquots may be transferred to individual empty test tubes. Open the canister containing the assay test strips. Remove one assay test strip from the canister and immediately recap the canister. Avoid touching the membrane surface in the middle of the strip with your fingers. Place the assay test strip in the tube containing 200 μl of the diluted specimen, with the arrows on the assay test strip point down (FIG. 1-12). Set a timer for 20 minutes, or note the time the assay test strip was added to the sample (FIG. 1-13). Read test result after 20 minutes (FIG. 1-14). Then, dispose of the strip, tube and swab in a bio-hazardous waste container (FIG. 1-15).

### Example 3

### Determining the Sensitivity, Specificity and Accuracy of the Kits

The sensitivity, specificity and accuracy of the oral fluid test are determined in this example. External validation trials of the Oral Fluid HIV Lateral tests began in April 2004 at the That Red Cross Anonymous HIV Clinic in Bangkok, Thailand and were completed in June 2004. 986 subjects who presented at the Anonymous HIV Clinic of the That Red Cross and were not currently under retroviral therapy underwent voluntary HIV antibody testing and counseling. The study was performed using sequential testing of subjects without prior knowledge of the results. In addition, 37 subjects who were known positive and receiving anti-retroviral therapy (ARV) also underwent voluntary HIV antibody testing and counseling. Subjects are given opportunity to voluntarily consent to provide additional samples for testing by these tests.

The reference methodology used at the Anonymous HIV Clinic was the Orgenics Rapid HIV-1/-2 Blood Test, Doublecheck™ II for initial screening. Reactive specimens from this test were confirmed using Bio-Rad GenScreen™ HIV-1/2 Version 2 ELISA and/or the Fujirebio Serodia®-HIV (HIV-1 only) Particle Agglutination Test.

The sensitivity is represented as the percentage obtained by dividing the number of positive in the reference test by the number of positive in the rapid oral fluid test. The specificity is represented as the percentage obtained by dividing the number of negative in the reference test by the number of negative in the rapid oral fluid test. And the accuracy is represented as the percentage obtained by dividing total number of subjects by the number of the consistent results between the rapid oral fluid test and the reference test. The results are shown in the following tables.

TABLE 1
rapid HIV-1/2 oral fluid test - recombinant HIV-1/synthetic HIV-2 peptide

| Number of | | | | Sensitivity | Specificity | Accuracy |
|---|---|---|---|---|---|---|
| subjects | The test utilized | Positive | Negative | (%) | (%) | (%) |
| 986 not on | Rapid oral fluid test | 355 | 631 | | | |
| ARV | Reference test | 355 | 631 | 100% | 100% | 100% |
| 37 on ARV | Rapid oral fluid test | 35 | 2 | | | |
| | Reference test | 37 | 0 | 94.6% | — | 94.6 |
| Total 1023 | Rapid oral fluid test | 390 | 633 | | | |
| | Reference test | 392 | 631 | 99.5% | 100% | 99.8% |

TABLE 2
rapid HIV-1/2 oral fluid test - synthetic HIV-1/synthetic HIV-2 peptide

| Number of | | | | Sensitivity | Specificity | Accuracy |
|---|---|---|---|---|---|---|
| subjects | The test utilized | Positive | Negative | (%) | (%) | (%) |
| 986 not on | Rapid oral fluid test | 353 | 633 | | | |
| ARV | Reference test | 355 | 631 | 99.4% | 100% | 100% |
| 37 on ARV | Rapid oral fluid test | 35 | 2 | | | |
| | Reference test | 37 | 0 | 94.6% | — | 94.6 |
| Total 1023 | Rapid oral fluid test | 388 | 635 | | | |
| | Reference test | 392 | 631 | 99.0% | 100% | 99.6% |

1. A lateral flow immunochromatography test strip for detecting an analyte in oral fluid, consisting essentially of a sample pad, a conjugate pad, a test zone and control zone pad made of at least one matrix material, wherein
the conjugate pad lies downstream of the sample pad, and is striped with a conjugate;
the test zone and control zone pad lies downstream of the conjugate pad, and contains the test zone and control zone, wherein
the test zone is immobilized with an specific binding reagent that specifically binding to the target analyte; and
the control zone is immobilized with a second capture reagent.

2. The test strip according to claim 1, wherein the analyte to be tested is selected from antibodies against antigens of infectious disease, hormones, growth factors, therapeutic drugs, drugs of abuse and products of the metabolism of drugs of abuse.

3. The test strip according to claim 1, wherein the matrix material is selected from inorganic powders, such as silica and alumina; glass fiber filter paper; natural polymeric material particularly cellulose-based materials, chromatographic paper; synthetic or modified naturally occurring polymers such as nitrocelluse, cellulose acetate, poly(vinyl chloride), polyacrylamide, crosslinked dextran, agarose; and the combination thereof.

4. The test strip according to claim 3, wherein the matrix material for the sample pad is glass fiber filter paper.

5. The test strip according to claim 3, wherein the matrix material for the conjugate pad is polyester material.

6. The test strip according to claim 3, wherein the matrix material for the test pad and control pad is nitrocellulose membrane.

7. The test strip according to claim 1, wherein the conjugate comprises a label conjugated to a first capture reagent that captures antibodies endogenous to the oral fluid.

8. The test strip according to claim 7, wherein the label is selected from colloidal gold particles; elemental or metal sol particles including selenium, silver, ferrite or carbon; other bead particles including colored latex, liposomes, and dye particles.

9. The test strip according to claim 7 or 8, wherein the label is colloidal gold particles.

10. The test strip according to claim 7, wherein the first capture reagent is selected from antibodies against IgG, IgM or IgA, protein A, protein G, and concanavalin A.

11. The test strip according to claim 8, wherein the first capture reagent is protein A.

12. The test strip according to claim 1, wherein the specific binding reagent is selected from antigens of infectious disease, hormones, growth factors, therapeutic drugs, drugs of abuse and products of the metabolism of drugs of abuse.

13. The test strip according to claim 12, wherein the antigen of infectious disease is recombinant or synthetic peptide representing the immunodominant region of HIV protein.

14. The test strip according to claim 13, wherein the HIV protein is HIV envelope protein.

15. The test strip according to claim 14, wherein the HIV envelope protein is selected from gp120 and gp41 of HIV-1 and gp36 of HIV-2.

16. The test strip according to claim 1, wherein the second capture reagent is selected from antibodies against IgG, IgM or IgA, protein A, protein G, and concanavalin A.

17. The test strip according to claim 16, wherein the antibody against IgG is goat anti-human IgG antibody.

18. A method for manufacturing a lateral flow immunochromatography test strip defined by any one of claims 1-17, comprising:
a) striping the conjugate onto the conjugate pad;
b) immobilizing the specific binding reagent onto the test zone of the test zone and control zone pad;
c) immobilizing the second capture reagent onto the control zone of the test zone and control zone pad;
d) blocking each of the pads with blocking agent; and
e) aligning the resulting pads in fluid communication relative to each other.

19. The method according to claim 18, wherein the conjugate is stabilized in a simple or complex sugar solution before striping.

20. The method according to claim 19, wherein the sugar solution contains sucrose, trehalose, potassium stannate and urea hydrogen peroxide.

21. The method according to claim 18, wherein the specific binding reagent is immobilized on the test zone using a biotin/strepavidin linker.

22. The method according to claim 18, wherein the specific binding reagent is immobilized on the control zone using a biotin/strepavidin linker.

23. The method according to claim 18, wherein the blocking agent contains detergents in nonionic, cationic, anionic and amphoteric forms; sugars including sucrose, fructose; or proteins including bovine serum albumin, whole animal serum, casein and nonfat dry milk.

24. The method according to claim 19, wherein the whole animal serum is fetal calf serum.

25. The method according to claim 19, wherein the whole animal serum is avian serum.

26. The method according to claim 21, wherein the avian serum is selected from goose serum, turkey serum, and chicken serum.

27. A lateral flow immunochromatography method for detecting an analyte in oral fluid, comprising:
(a) collecting oral fluid with a collector separated from the lateral flow immunochromatography test strip defined by any one of claims 1-17;
(b) plunging the collector in a volume of sample buffer to release the oral fluid into the buffer to get the mixture;
(c) placing the lateral flow immunochromatography test strip defined by any one of the proceeding claims into the mixture; and
(d) determining the validity of the test by observing the presence of the signal in the control zone, and determining the presence of the analyte by observing the presence of the signal in the test zone within 15-60 min from start of the test.

28. The method according to claim 27, wherein the collector in step (a) is an untreated polyester swab.

29. The method according to claim 28, wherein the swab is Texwipe Large Alpha Swab TX714A.

30. The method according to claim 27, wherein the sample buffer is potassium phosphate pH 7.2+/−0.2 buffered solution.

31. The method according to claim 30, wherein the solution further contains 0.15 M sodium chloride, 0.1% Triton X-100, 15% heat inactivated chicken serum, 30 μg/ml Avidin, 0.2% Tween 80, 0.2% Tetronic T-904 and 0.0.285% (active ingredient) ProClin 950.

32. The method according to claim 27, wherein the volume of the sample buffer is 1000 μl.

33. The method according to claim 27, wherein the method further comprises a step of taking out an aliquot of mixture between step (b) and step (c).

34. The method according to claim 33, wherein the volume of the aliquot is 200 μl.

35. The method according to claim 27, wherein the signal is a colored line.

36. The method according to claim 35, wherein the colored line is a reddish line.

37. The method according to claim 27, wherein the signal is observed within 20-45 minutes.

38. A kit for detecting an analyte in oral fluid, comprising at least one lateral flow immunochromatography test strip defined by any one of claims 1-17.

39. The kit according to claim 38, wherein the test strip is closed in a desiccated container.

40. The kit according to claim 38, wherein the kit further comprises a sample buffer, and at least one collector for collecting oral fluid.

41. The kit according to claim 40, wherein the sample buffer is potassium phosphate pH 7.2+/−0.2 buffered solution.

42. The kit according to claim 41, wherein the solution further contains 0.15 M sodium chloride, 0.1% Triton X-100, 15% heat inactivated chicken serum, 30 μg/ml Avidin, 0.2% Tween 80, 0.2% Tetronic T-904 and 0.0285% (active ingredient) ProClin 950.

43. The kit according to claim 40, wherein the collector is an untreated polyester swab.

44. The kit according to claim 43, wherein the swab is Texwipe Large Alpha Swab TX714A.

45. The kit according to any one of claim 38 to 44, wherein the kit further comprises vials containing positive and negative control for quality testing the test strip.


SEQUENCE LISTING (5 sequences)

SEQ ID NO: 1 - protein, 39 aa, Artificial
  Description of Artifical Sequence biotinylated single-lysine linker HIV-1 (gp41 peptide)
XILAVERYLKDQQLLGIWGCSGKLICTTAVPWNASGKLI

SEQ ID NO: 2 - protein, 22 aa, Artificial
  Description of Artificial Sequence biotinylated HIV-1 (gp120 peptide)
XTEPLGVAPTKAKRRVVQREKR

SEQ ID NO: 3 - protein, 35 aa, Artificial
  Description of Artificial Sequence biotinylated single-lysine linkder HIV-2 (gp36 peptide)
XVTAIEKYLQDQARLNSWGCAFRQVCHTVPWVNDX

SEQ ID NO: 4 - protein, 40 aa, Artificial
  Description of Artificial Sequence biotinylated single-lysine linkider HIV-2 (gp41 peptide)
XKILAVERYLKDQQLLGIWGCSGKLICTTAVPWNASGKLI

SEQ ID NO: 5 - protein, 37 aa, Artificial
  Description of Artificial Sequence biotinylated double-lysine linker HIV-2 (gp36 peptide)
XKVTAIEKYLQDQARLDSWGCAFRQVCHTTVPWVNDX